(19) United States
(12) Patent Application Publication
(10) Pub. No.: US 2003/0069618 A1
(43) Pub. Date: Apr. 10, 2003

(54) Method, kit and device for the treatment of cosmetic skin conditions

(76) Inventors: Edward Dewey Smith III, Mason, OH (US); John Erich Oblong, Loveland, OH (US); Jonathan Todd Samuel, West Chester, OH (US); Donald Lynn Bissett, Hamilton, OH (US); Charles Carson Bascom, Hamilton, OH (US); Gary Robert Kelm, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/132,045

(22) Filed: Apr. 25, 2002

Related U.S. Application Data
(60) Provisional application No. 60/286,843, filed on Apr. 26, 2001.

(51) Int. Cl. A61N 1/00; A61F 7/00; A61F 7/12; A61F 2/00

(52) U.S. Cl. 607/100; 607/96; 607/91

(57) ABSTRACT

The present invention concerns a method and a kit, for the treatment of a selected area of the skin and/or subcutaneous tissue, and in particular for the cosmetic treatment of skin conditions such as regional fat deposits including cellulite. The method comprises heating the selected area to a sustained skin temperature of about 32 to about 50° C. for a desired period of time, using a device comprising either a heat source capable of conductively heating the selected area or, alternatively, an infra-red source for emitting infra-red with a wavelength from about 700 nm to about 15000 nm. The method also comprises administering, simultaneously or sequentially in either order, a composition containing an active agent selected from the group comprising a skin active, a nutrient absorption suppressant or a thermogenic agent, preferably a metabolic stimulating agent, more preferably a lipolytic agent, or a mixture thereof, the composition being either a topical composition for application to the selected area, or an area adjacent thereto, or an oral composition.

### CROSS REFERENCE TO RELATED APPLICATIONS

This application claims the benefit of U.S. Provisional Application No. 60/286,843, filed Apr. 26, 2001.

### FIELD OF THE INVENTION

The present invention relates to a method for the treatment of cosmetic skin conditions including regional fat deposits such as cellulite using heat, with either a topical or an oral composition or both. The method comprises heating the selected area to a sustained skin temperature of about 32 to about 50° C. for a desired period of time, using a device comprising either a heat source capable of conductively heating the selected area or, alternatively, an infra-red source for emitting infra-red with a wavelength from about 700 nm to about 15000 nm; and administering, simultaneously or sequentially in either order, a composition containing an active agent selected from the group comprising a skin active, a nutrient absorption suppressant or a thermogenic agent, preferably a metabolic stimulating agent, more preferably a lipolytic agent, or a mixture thereof, the composition being either a topical composition for application to the selected area, or an area adjacent thereto, or an oral composition.

The invention further relates to a kit for the treatment of cosmetic skin conditions including regional fat deposits such as cellulite. The kit includes:

i. a device for maintaining a sustained skin temperature of about 32 to about 50° C. for a desired period of time, the device comprising either a heat source capable of conductively heating the selected area or, alternatively, an infra-red source for emitting infra-red with a wavelength from about 700 nm to about 15000 nm;

ii. a composition containing an active agent selected from the group comprising a skin active; a nutrient absorption suppressant; or a thermogenic agent, preferably a lipolytic agent, or a mixture thereof, the composition being either a topical composition for application to the selected area, or an area adjacent thereto, or an oral composition; and

iii. instructions for their simultaneous or sequential use in either order.

### BACKGROUND TO THE INVENTION

As we age, and as a normal course of hormonal fluctuations, environmental influences and individual genetic tendencies, skin elasticity is gradually reduced. At the same time, lean tissue mass decreases and adipose tissue increases. Generally speaking, adipose tissue tends to concentrate the body's fat stores in a few regional sites of the body, such as the mid-section, the thighs and buttocks, and/or the back of the arms. In some regions, especially the legs, bulging of fat chambers near the skin's surface can cause dimpling of the skin at the attachment points of the skin's underlying structural fibrous strands. This regional fat deposit is termed cellulite and it occurs most often on the thighs, hips, waist, buttocks and upper arms of women. Cellulite is a cosmetic rather than a medical condition.

The dimpling of the skin affected by regional fat deposits including cellulite is also known as the “orange peel” effect, which affects women of all ages and sizes, although it is generally more prevalent in women who are overweight to some degree. In a society that is increasingly concerned with image, women have resorted to many methods to try to rid themselves of regional fat deposits including cellulite.

To date, many creams for the treatment of regional fat deposits including cellulite have been available on the market. Relatively expensive to buy, their results are often minimal and short-lived. Dry-brushing is another method suggested for the treatment of regional fat deposits including cellulite, which involves the frequent brushing of oneself with relative vigour with the bristles of a suitable brush. The method of dry-brushing, however, leaves the skin feeling relatively uncomfortable and raw, and it also often has a minimal effect on the regional fat deposits including cellulite.

There has been a desire in recent years to provide a different method of treating regional fat deposits including cellulite which is both effective and relatively painfree.

Regional fat deposits including cellulite is not the only cosmetic condition that concerns women. Stretch marks are another example of a cosmetic condition which affects not only women, but also men. Stretch marks can form at various stages of a person's life, for example, at puberty, during pregnancy in the case of women, or generally when a person gains a substantial amount of weight. Stretch marks are most commonly found on the thighs, buttocks and abdomen, but also quite frequently appear on other areas, the upper arms for example. The stretch marks appear as generally purple blemishes on the skin, generally quite long and thin, with a length dependable on the position of the body on which they are found and the reason for the formation of the stretch marks. Over time, the stretch marks fade in colour and eventually have a silver appearance. It is virtually impossible to rid oneself of stretch marks using conventional methods. Loss of weight will result in their appearance being less noticeable but they are still present on the skin. Creams are available on the market which claim to reduce the appearance of the stretch marks, but the effect of these creams are generally minimal and short-lived, similar to the effects of the creams for the treatment of regional fat deposits including cellulite.

U.S. Pat. No. 5,358,503, which is incorporated herein by reference, discloses an apparatus for simultaneous or selective treatment of an area of the skin using photoenergy and therapeutic heat. Specifically, the apparatus includes a plurality of resisters to heat a plurality of diodes, which act as heat sinks, so that the treatment area of the skin receives light and thermal treatment simultaneously. The apparatus is a substantially rigid and non-flexible device.

U.S. Pat. No. 6,187,029, which is incorporated herein by reference, discloses a photo-thermal treatment device for applying light and heat stimulation to a surface of the human body, for musculoskeletal pain relief, cosmetic rejuvenation and accelerated healing of open and closed wounds. The device is a substantially rigid and non-flexible device.

Neither U.S. Pat. No. 5,358,503 nor U.S. Pat. No. 6,187,029 disclose or suggest a sustained skin temperature of 32 to 50° C. In addition, neither U.S. Pat. No. 5,358,503 nor U.S. Pat. No. 6,187,029 are adapted for wearing by a user in that neither document discloses or suggests adaptation to a substantially flexible device/apparatus arranged for conforming to a treatment area of the skin.

U.S. Pat. No. 4,829,987, which is incorporated herein by reference, concerns a method for treating the human body to reduce body dimensions and minimise the undesirable appearance of cellulite. The method involves combining a mineral solution at 150° F. (65.6° C.) with a body wrap material which has been pre-warmed to about 150° F. (65.5° C.) and then wrapping the soaked body wrap material on a patient's body for a period of 60 to 70 minutes. U.S. Pat. No. 4,829,987 does not disclose or suggest a sustained skin temperature of 32 to 50° C.

U.S. Pat. No. 4,741,338, which is incorporated herein by reference, concerns a thermoelectric apparatus to, for example, diminish or remove superfluous flesh from the abdomen. The apparatus can be provided in the form of a belt including a plurality of thermo-modules, each of which is heated to a specific temperature corresponding to the sum of normal temperature and 20-25° C. to a maximum of 100° C. during the heating cycle. U.S. Pat. No. 4,741,338 neither discloses nor suggests a sustained skin temperature of 32 to 50° C.

EP 695,559, which is incorporated herein by reference, relates to multifunctional equipment for beauty treatment such as cellulitis, which may include electrical resistances for thermotherapy. EP 695,559 neither discloses nor suggests a sustained skin temperature of 32 to 50° C.

FR 2732216, which is incorporated herein by reference, relates to treatment of cellulite marks by applying a heating and then a cooling substance to the surface of a body and then compressing. The heating substance is at a temperature greater than room temperature. FR 2732216 neither discloses nor suggests a sustained skin temperature of 32 to 50° C.

None of the known thermotherapy methods and kits require a sustained skin temperature of 32 to 50° C. In addition, none of the known thermotherapy methods and kits use a simultaneously or sequentially applied composition, in which the composition is either a topical composition or an oral composition or both.

Furthermore, none of the known thermotherapy methods and kits are adapted for domestic use or for unsupervised clinic use, preferably by additionally comprising a portable power source.

Surprisingly, it has been found that the use of therapeutic heat with a topical and/or an oral composition on a skin treatment area results in an improved response in the treatment of cosmetic skin conditions such as regional fat deposits including cellulite. In addition, the use of a device, which is optionally wearable, for generating such therapeutic heat, to be used in combination with a topical and/or an oral composition, allows the kit and method of the present invention to be used effectively yet safely in a domestic or non-clinical enviromnent.

It is an object of the present invention to provide a kit for improved treatment of skin and/or subcutaneous tissue, and in particular for the treatment of cosmetic skin conditions such as regional fat deposits including cellulite in a selected area, using therapeutic heat with a topical and/or an oral composition.

It is a further object of the present invention to provide a method which enables the efficient treatment of a selected area of the skin and/or subcutaneous tissue, and in particular the cosmetic treatment of skin conditions such as regional fat deposits including cellulite, using therapeutic heat with an oral and/or a topical composition.

It is a still further object of the present invention to provide a kit or a method which uses therapeutic heat with a topical and/or an oral composition, which kit or method is suitable for domestic use or unsupervised use in clinics. These, and other objects of this invention, will become apparent in the light of the following disclosure.

### SUMMARY OF THE INVENTION

The present invention relates to a method for the treatment of a selected area of the skin and/or subcutaneous tissue, and in particular for the cosmetic treatment of skin conditions such as regional fat deposits including cellulite, the method comprising heating the selected area to a sustained skin temperature of about 32 to about 50° C. for a desired period of time, using a device comprising either a heat source capable of conductively heating the selected area or, alternatively, an infra-red source for emitting infra-red with a wavelength from about 700 nm to about 15000 nm; and administering, simultaneously or sequentially in either order, a composition containing an active agent selected from the group comprising a skin active, a nutrient absorption suppressant or a thermogenic agent, preferably a metabolic stimulating agent, more preferably a lipolytic agent, or a mixture thereof, the composition being either a topical composition for application to the selected area, or an area adjacent thereto, or an oral composition.

The present invention also relates to a kit for the treatment of skin and/or subcutaneous tissue, and in particular for the treatment or prevention of regional fat deposits including cellulite in a selected area, the kit including:

i. a device for maintaining a sustained skin temperature of about 32 to about 50° C. for a desired period of time, the device comprising either a heat source capable of conductively heating the selected area or, alternatively, an infra-red source for emitting infra-red with a wavelength from about 2000 nm to about 15000 nm;

ii. a composition containing an active agent selected from the group comprising a skin active; a nutrient absorption suppressant; or a thermogenic agent, preferably a lipolytic agent, or a mixture thereof, the composition being either a topical composition for application to the selected area, or an area adjacent thereto, or an oral composition; and

iii. instructions for their simultaneous or sequential use in either order.

The present invention further relates to a device for the treatment of skin and/or subcutaneous tissue, and in particular for the treatment or prevention of regional fat deposits including cellulite in a selected area, the device being adapted for maintaining a sustained skin temperature of about 32 to about 50° C. for a desired period of time, the device comprising either a heat source capable of conductively heating the selected area or, alternatively, an infra-red source for emitting infra-red with a wavelength from about 700 nm to about 15000 nm; the device also being adapted for domestic use or unsupervised clinic.

### BRIEF DESCRIPTION OF THE FIGURES

FIG. 1 is a schematic drawing of the heating pad of Example 2.

### DETAILED DESCRIPTION OF THE INVENTION

All publications cited herein are hereby incorporated by reference in their entirety, unless otherwise indicated.

As used herein, the term “subcutaneous tissue” means tissue lying beneath the skin and includes adipose tissue and subcutaneous fat.

As used herein, the term “regional fat deposits” means areas of excessive fat, of which cellulite is an example, and excess fatty tissue.

As used herein, the term “cellulite” means deposits of fat, which generally do not respond to dieting and exercise.

As used herein, the term “light” means monochromatic, dichromatic or multichromatic electromagnetic radiation in the visible, red or infrared ranges. The use of light in the treatment of cosmetic skin conditions and/or subcutaneous tissue, and in particular cellulite in human skin, comprises exposing the area of treatment to a source of electromagnetic radiation, preferably having a wavelength of from approximately 600 nanometers to approximately 1100 nanometers. The electromagnetic radiation may be applied by means of one or more LED's, one or more lasers, one or more light bulbs, or any other suitable source of electromagnetic radiation. The electromagnetic radiation may be coherent or non-coherent, pulsed or continuous, or combinations thereof.

As used herein, the term “electrotherapy” means the application of either a static or active electric current to the treatment site, and may include such applications as muscular electrical nerve stimulation (MENS), transcutaneous electrical nerve stimulation (TENS), and iontophoresis, but is not intended to be limited thereto.

As used herein, the term “static magnet” means a magnet with a static magnetic field having an intensity of from 100 to 2000 gauss, the magnet, in use, imparting a monopolar or bipolar magnetic polarity to the body of a user. The use of static magnets in the treatment of cellulite preferably involves exposing an area of skin and/or subcutaneous tissue, and in particular cellulite in human skin, to the static magnetic field thereof.

As used herein, the term “active massage” means the stimulation of biological tissue by physical or mechanical means. Massaging tissue involves application of stress from outside the tissue, either compression or tension (both are beneficial). The stress can be applied randomly or directionally, for example directed in the direction of the lymph flow. Non-limiting examples of massaging devices are percussive, roller, pinching and vacuum massagers, and combinations thereof. Massage to cellulite skin has the following benefits:

1. Stimulating flow of lymph

2. Increasing blood flow

3. Stretching the connective tissue fibers

4. Remodelling the dermal interface with the subcutaneous adipose tissue

5. Promoting cellular activity via stress-orientation

As used herein, the term “laser” means light amplification by stimulated emission of radiation.

As used herein, the term “topical” means designed for or involving local application and action.

As used herein, the term “ultrasound” means pressure waves having a frequency of at least 16 kHz, preferably at least 20 kHz, the application of which may be either continuous or pulsed. Pulsed ultrasound is effectively a train of pulses. For example, ultrasound can be delivered in an “on-off” mode, where the unit pulses on for 0.2 seconds, then off for 0.8 seconds, with this cycle being repeated indefinitely. Pulsing is typically used for high energy input uses. The “off” time allows heat that may have built up in one area to diffuse away, such that no localised hot spots result. For the present invention, pulsing is acceptable and will produce the desired results, but continuous wave ultrasound is preferred.

As used herein, the term “compression” means the application of static pressure by wrapping or otherwise, increasing the pressure in the tissues.

As used herein, the term “therapeutic heat” means the application of heat to the skin to increase skin and tissue temperatures into the therapeutic range of 32 to 50° C. for a period sufficient to promote a therapeutic benefit as defined in this application. Without being limited by theory, it is believed that therapeutic heat is beneficial for treatment of cosmetic skin conditions, in particular treatment of conditions exhibiting an excess of underlying adipose tissue, and in particular for the treatment of cellulite. Many cosmetic skin conditions can result from a deficit in local blood supply or can be mitigated by increased blood flow. In the condition of cellulite, a reduction in local blood supply to the tissues results from increased pressure on the tissues due to upwards pressure from excess underlying adipose tissue, as well as, from deposition of plaque-like substances (hypothesised to be proteoglycans) that clog the arterioles and venous capillaries. The application of therapeutic heat to the tissues results in a rapid increase in blood supply to the tissues due to the body's thermoregulatory mechanism. Blood perfusion to the skin can be increased by several-fold, depending on factors such as the heat application time, temperature, and the basal condition of the tissue. The increased blood perfusion flushes the capillaries and arterioles, resupplying the tissues with needed, newly oxygenated blood, and enhancing lymphatic drainage.

In addition to stimulating circulation, the increased local temperature in the tissues increases metabolic activity. It is well established that, for every 10° C. increase in temperature, metabolic activity in cells and tissues approximately doubles. Increased metabolic activity is beneficial to promote activity in the adipocytes, such as lipolysis and the burning of energy that accompany metabolic activity, especially thermogenic losses.

Particularly beneficial effects are realized in conditions with excess underlying adiposity when the body or the target tissues are simultaneously encouraged into a condition of enhanced lipolytic activity at the expense of other possible fuel sources, for example, by application of a topical composition comprising at least one agent selected from thermogenic agents, preferably lipolytic agents, or a mixture thereof, or by oral ingestion of at least one agent selected from thermogenic agents, preferably lipolytic agents, which promote a general lipolytic condition in the body; or an nutrient absorption suppressant, or a mixture thereof. In particular, it is believed that stimulation of systemic lipolysis with simultaneous or sequential application of therapeutic heat locally can encourage the body to regional lipolysis in the cosmetic site of interest, e.g., cellulite. Beneficial effects are also realised if the topical composition comprises at least one skin active to simultaneously improve the skin condition and strengthen the dermis and epidermis.

Finally, in addition to the aforementioned benefits of therapeutic heat, it is believed that certain levels of heat can stimulate cellular processes via a heat shock mechanism. Classical heat shock and apoptosis (programmed cell death) are known to occur in the temperature range of about 44° C. and higher. It is believed that temperatures between about 38° C. and 50° C. for brief periods of time can encourage cell and tissue growth and repair processes via liberation of heat shock proteins within cells, whilst remaining short of the temperatures necessary for general apoptosis such as might be utilized therapeutically, for example, for tumor destruction.

It should be recognized that the temperature at the skin surface and the temperature in the interior of the skin are not the same because the interior of the skin is separated from the heat source via the stratum corneum layer; and conduction and blood perfusion remove heat, at the same time as heat is being applied to the skin surface. In the case of heat by conduction alone, when temperature is elevated above the body core temperature (about 37.0° C.), the skin surface temperature is always the maximum temperature.

To provide a beneficial therapeutic effect, it has been discovered that maintaining a sustained skin temperature of from about 32° C. to about 50° C., preferably from about 32° C. to about 45° C., more preferably from about 34° C. to about 45° C., most preferably from about 35° C. about 45° C., still most preferably from about 36° C. to about 44° C., for a period of from about twenty seconds to about twenty-four hours, preferably from about five minutes to about sixteen hours, more preferably from about ten minutes to about twelve hours, most preferably from about thirty minutes to about eight hours, wherein the maximum skin temperature and the length of time of maintaining the skin temperature at the maximum skin temperature may be appropriately selected by a person needing such treatment, such that the desired therapeutic benefits are achieved without any adverse events, such as skin bums, which may be incurred by using a high temperature for a long period of time, is substantially beneficial for treatment of cosmetic skin conditions, in particular treatment of conditions exhibiting an excess of underlying adipose tissue, and in particular for the treatment of cellulite.

As used herein, the term “wearable device”, which includes the term “sleeve”, means a substantially flexible section of material in the form of, for example, a wrap, patch, cuff or a bandage which may be placed on/confirm to or which may be held adjacent, a selected area of the body. Such a wrap, patch, cuff or bandage may be formed from a substrate, preferably a disposable substrate. The sleeve may, in addition, be dimensioned and adapted to apply compression. The sleeve in the form of a wrap, patch, cuff or bandage may be held in place by the use of straps or fasteners. For example, one side of the sleeve may be connected to the other side of the sleeve, using buttons, Velcro (Trade Mark) or the like. Alternatively, the sleeve may be adapted to form a shape which is specifically designed to fit on an arm, leg, buttocks, stomach or other selected body part. The sleeve may therefore be in the form of a garment such as a sock, trousers, shorts or the like. The material which forms the sleeve is generally flexible and may also have a degree of elasticity. The flexible nature of the sleeve enables the sleeve to conform to the desired shape, and, for example, to enable the sleeve to be pulled up over the selected area of the body. The optionally elastic nature of the sleeve facilitates the sleeve to fit the selected body part in a suitably tight yet comfortable manner.

Cosmetic Skin Conditions

The term “cosmetic skin conditions”, as used herein, includes signs of skin ageing and regional fat deposits including cellulite. “Signs of skin ageing” include, but are not limited to, all outward visibly and tactilely perceptible manifestations as well as any other macro or micro effects due to skin ageing. Such signs may be induced or caused by intrinsic or extrinsic factors, e.g., chronological ageing and/or environmental damage (e.g., sunlight, UV, smoke, ozone, pollutants, stress, etc.). These signs may result from processes which include, but are not limited to, the development of textural discontinuities such as wrinkles, including both fine superficial wrinkles and coarse deep wrinkles, skin lines, facial frown lines, expression lines, rhytides, dermatoheliosis, photodamage, premature skin ageing, crevices, bumps, pits, large pores (e.g., associated with adnexal structures such as sweat gland ducts, sebaceous glands, or hair follicles), “orange peel” skin appearance, dryness, scaliness, flakiness and/or other forms of skin unevenness or roughness; excess skin oil problems such as over-production of sebum, oiliness, facial shine, foundation breakthrough; abnormal desquamation (or exfoliation) or abnormal epidermal differentiation (e.g., abnormal skin turnover) such as scaliness, flakiness, keratoses, hyperkeratinization; inadequate skin moisturization (or hydration) such as caused by skin barrier damage, environmental dryness; loss of skin elasticity (loss and/or inactivation of functional skin elastin) such as elastosis, sagging (including puffiness in the eye area and jowls), loss of skin firmness, loss of skin tightness, loss of skin recoil from deformation; non-melanin skin discoloration such as undereye circles, blotching (e.g., uneven red coloration due to, e.g., rosacea), sallowness (pale colour), discoloration caused by telangiectasia; melanin-related hyperpigmented (or unevenly pigmented) skin regions; post-inflammatory hyperpigmentation such as that which occurs following an inflammatory event (e.g., an acne lesion, in-grown hair, insect/spider bite or sting, scratch, cut, wound, abrasion, and the like); atrophy such as, but not limited to, that associated with ageing or steroid use; other histological or microscopic alterations in skin components such as ground substance (e.g., hyaluronic acid, glycosaminoglycans, etc.), collagen breakdown and structural alterations or abnormalities (e.g., changes in the stratum corneum, dermis, epidermis, the skin vascular system such as telangiectasia); tissue responses to insult such as itch or pruritus; and alterations to underlying tissues (e.g., subcutaneous fat, cellulite, muscles, trabeculae, septae, and the like), especially those proximate to the skin.

Topical Compositions: Carriers

It is envisaged that topical compositions may perform pharmaceutical and/or cosmetic functions.

The topical carrier compositions of the present invention can comprise a carrier. The carrier should be “dermatologically acceptable”, which means that the carrier is suitable for topical application to the skin, has good aesthetic properties, is compatible with the remaining components, and will not cause any untoward safety or toxicity concerns. A safe and effective amount of carrier is from about 50% to about 99.99%, preferably from about 80% to about 99.9%, more preferably from about 90% to about 98%, most preferably from about 90% to about 95% of the composition.

The carrier can be in a wide variety of forms. For example, emulsion carriers, including, but not limited to, oil-in-water, water-in-oil, water-in-oil-in-water, and oil-in-water-in-silicone emulsions, are useful herein. These emulsions can cover a broad range of viscosities, e.g., from about 100 cps to about 200,000 cps (at room temperature). These emulsions can also be delivered in the form of sprays using either mechanical pump containers or pressurised aerosol containers using conventional propellants. These carriers can also be delivered in the form of a mousse. Other suitable topical carriers include anhydrous liquid solvents such as oils, alcohols, and silicones (e.g., mineral oil, ethanol, isopropanol, dimethicone, cyclomethicone, and the like); aqueous-based single phase liquid solvents (e.g., hydro-alcoholic solvent systems); and thickened versions of these anhydrous and aqueous-based single phase solvents (e.g., where the viscosity of the solvent has been increased to form a solid or semi-solid by the addition of appropriate gums, resins, waxes, polymers, salts, and the like). Examples of topical carrier systems useful in the present invention are described in the following four references all of which are incorporated herein by reference in their entirety: “Sun Products Formulary” Cosmetics & Toiletries, vol. 105, pp. 122-139 (December 1990); “Sun Products Formulary”, Cosmetics & Toiletries, vol. 102, pp. 117-136 (March 1987); U.S. Pat. No. 4,960,764 to Figueroa et al., issued Oct. 2, 1990; and U.S Pat. No. 4,254,105 to Fukuda et al., issued Mar. 3, 1981.

A further discussion of suitable carriers is found in U.S. Pat. No. 5,605,894 to Blank et al, and U.S. Pat. No. 5,681,852 to Bissett, both of which are herein incorporated by reference in their entirety.

Topical Compositions: Skin Actives

The compositions of the present invention may optionally comprise one or more skin actives. By the term “skin active” is meant an agent that promotes the growth of healthy skin tissue by, for example, supporting tissue revascularisation. Non-limiting examples of such skin actives include vitamin B3 compounds such as those described in WO 97/39733, published Oct. 30, 1997, to Oblong et al., herein incorporated by reference in its entirety; hydroxy acids such as salicylic acid; anti-oxidants/radical scavengers such as tocopherol and esters thereof; metal chelators, especially iron chelators; retinoids such as retinol, retinyl palmitate, retinyl acetate, retinyl propionate, and retinal; N-acetyl-L-cysteine and derivatives thereof; hydroxy acids such as glycolic acid; keto acids such as pyruvic acid; benzofuran derivatives; anti-cellulite agents (e.g., xanthines such as caffeine, theophylline); niacinamide, which promotes healthy cell growth in the dermis; polycyclic compounds such as triterpenoids (e.g., betulinic acid); and sterols such as stigmasterol. Mixtures of any of the above mentioned skin actives may also be used. A more detailed description of these actives is found in U.S. Pat. No. 5,605,894 to Blank et al (previously incorporated by reference).

Other conventional active ingredients, or mixtures thereof, may also be included. These include exfoliation or desquamatory agents such as zwitterionic surfactants; sunscreens such as 2-ethylhexyl-p-methoxycinnamate, 4,4′-t-butyl methoxydibenzoyl-methane, octocrylene, phenyl benzimidazole sulfonic acid; sun-blocks such as zinc oxide and titanium dioxide; anti-inflammatory agents; depilatory agents (e.g., sulfhydryl compounds); skin lightening agents (e.g., arbutin, kojic acid, hydroquinone, ascorbic acid and derivatives such as ascorbyl phosphate salts, placental extract, and the like); moisturizing agents; anti-microbial agents; anti-androgens; and skin protectants. Ultraviolet absorbing agents, often described as sunscreening agents, can be present in a concentration in the range of between about 1% and about 12% by weight, based on the total weight of composition. Preferably, the UV absorbing agents constitute between about 2% and 8% by weight. More preferably, the UV absorbing agents can be present in the composition in a concentration range of between about 4% and about 6% by weight. Of the ultraviolet absorbing agents suitable for use herein, benzophenone-3, octyl -dimethyl PABA (Padimate O), Parsol MCX, and mixtures thereof are particularly preferred. Also useful in topical compositions of the present invention are sunless tanning agents including dihydroxyacetone, glyceraldehyde, indoles and their derivatives, and the like. These sunless tanning agents can also be used in combination with the sunscreen agents.

An optional skin active of the topical compositions of the present invention is a flavonoid compound—an aromatic compound having two substituted benzene rings connected by a chain of three carbon atoms and an oxygen bridge. Flavonoids are broadly disclosed in U.S. Pat. No. 5,686,082 and U.S. Pat. No. 5,686,367, both of which are herein incorporated by reference. Flavonoids suitable for use in the present invention are flavanones selected from the group consisting of unsubstituted flavanones, mono-substituted flavanones, and mixtures thereof; chalcones selected from the group consisting of unsubstituted chalcones, mono-substituted chalcones, di-substituted chalcones, tri-substituted chalcones, and mixtures thereof; flavones selected from the group consisting of unsubstituted flavones, mono-substituted flavones, di-substituted flavones, and mixtures thereof; one or more isoflavones; coumarins selected from the group consisting of unsubstituted coumarins, mono-substituted coumarins, di-substituted coumarins, and mixtures thereof, chromones selected from the group consisting of unsubstituted chromones, mono-substituted chromones (including 3-formyl chromone), di-substituted chromones, and mixtures thereof; one or more dicoumarols; one or more chromanones; one or more chromanols; isomers (e.g., cis/trans isomers) thereof, and mixtures thereof. By the term “substituted” as used herein means flavonoids wherein one or more hydrogen atom of the flavonoid has been independently replaced with hydroxyl, C₁-C₈ alkyl, C₁-C₄ alkoxyl, O-glycoside, and the like or a mixture of these substituents.

The flavonoid compounds can be synthetic materials or obtained as extracts from natural sources (e.g., plants). The naturally sourced material can also further be derivatized (e.g., an ester or ether derivative prepared following extraction from a natural source). Flavonoid compounds useful herein are commercially available from a number of sources, e.g., Indofine Chemical Company, Inc. (Somerville, N.J.), Steraloids, Inc. (Wilton, N.H.), and Aldrich Chemical Company, Inc. (Milwaukee, Wis.). Preferred naturally sourced materials include kava root (standardised to give a kavalactone content of about 30% by wt and containing the full spectrum of lactones found in the kava plant) and green tea solids containing the full range of green tea polyphenols (i.e. catechins and epicatechins)—such materials may, optionally, be ingested as part of an oral composition.

Mixtures of flavonoid compounds may also be used.

Other suitable additives or skin actives are discussed in further detail in WO 97/39733, published Oct. 30, 1997, to Oblong et al, previously incorporated by reference in its entirety.

Optional Components: Topical Compositions

Compositions optionally comprise a pigment or mixture of pigments. The pigment used herein must be compatible with any acidic skin care active which may be present in the composition and have excellent overall colour stability. Suitable pigments for use herein can be inorganic and/or organic. Also included within the term pigment are materials having a low colour or lustre such as matte finishing agents, and also light scattering agents. Examples of suitable pigments are iron oxides, rutile titanium dioxide, anatase titanium dioxide, ferric oxide, ferrous oxide, chromium oxide, chromium hydroxide, manganese violet, acylglutamate iron oxides, ultramarine blue, D&C dyes, carmine, and mixtures thereof. Depending upon the type of make-up composition, e.g. foundation or blusher, a mixture of pigments will normally be used.

If the composition is a foundation, then the foundation composition can also include at least one matte finishing agent. The function of the matte finishing agent is to hide skin defects and reduce shine. Such cosmetically acceptable inorganic agents, i.e., those included in the CTFA Cosmetic Ingredient Dictionary, Third Ed., as silica, hydrated silica, silicone-treated silica beads, mica, talc, polyethylene, titanium dioxide, bentonite, hectorite, kaolin, chalk, diatomaceous earth, attapulgite zinc oxide and the like may be utilized.

An optional component of the topical compositions herein is a humectant or mixture of humectants, which can act as skin conditioners and are, therefore, to be considered as skin actives. The humectant or mixture of humectants herein is optionally present in an amount of from about 0.1% to about 30% preferably from about 1% to about 25%, and more preferably from about 1% to about 10% by weight of composition. Other conventional skin care product humectants may also be included in the compositions of the present invention. For example, urea, guanidine and mixtures thereof may be used. Glycerine is a preferred humectant.

The topical compositions herein can additionally comprise an emollient. Emollients suitable for the compositions of the present invention include natural and synthetic oils selected from mineral, vegetable, and animal oils, fats and waxes, such as petrolatum, fatty acid esters, fatty alcohols, alkylene glycol and polyalkylene glycol ethers and esters, fatty acids and mixtures thereof.

Another optional component herein is one or more additional chelating agents, preferably in the range of from about 0.02% to about 0.10% by weight, based on the total weight of the composition. Preferably, the chelating agent is present in a concentration in the range of between about 0.03% and about 0.07% by weight, based on the total weight of the composition. Among the chelating agents that may be included in the composition is tetrasodium EDTA.

Another optional but preferred component of the topical composition is one or more preservatives. The preservative concentration in the composition, based on the total weight of that composition, is in the range of between about 0.05% and about 0.8%, preferably between about 0.1% and about 0.3%. Suitable preservatives for use herein include sodium benzoate and propyl paraben, and mixtures thereof.

Oral Compositions

Oral compositions are generally intended either to induce satiety/promote nutrient malabsorption and thereby indirectly enhance thermogenesis and/or to directly enhance thermogenesis to consume fat/calories and/or stimulate metabolic activity in general and lipolytic activity in particular.

Oral dosage forms are alternative compositions for use in the present invention and these include the known forms for such administration, for example tablets, capsules, granules, syrups and aqueous or oil suspensions. Any carriers known in the art for oral application compositions may be used. For solid form preparations, such as, for example, powders, tablets, disbursable granules and capsules, a solid carrier may be one or more substances such as diluents, flavoring agents, solubilizers, lubricants, suspending agents, binders, tablet disintegrating agents, encapsulating materials and the like. Suitable carrier materials may include, for example, magnesium carbonate, calcium carbonate, sodium bicarbonate, magnesium stearate, calcium stearate, talc, lactose, sugar, pectin, dextrin, starch, tragacanth, cellulose derivatives, methyl cellulose, sodium carboxymethyl cellulose, a low-melting wax, cocoa butter, alginates, gelatin, polyvinyl pyrrolidone, polyethyl glycols, quaternary ammonium compounds and the like.

Tablets may be prepared from an active agent (nutrient absorption suppressant(s) and/or thermogenic agent(s)) or a mixture thereof (see below), with fillers, for example, calcium phosphate; disintegrating agents, for example, maize, starch; lubricating agents, for example, magnesium stearate; binders, for example, microcrystalline cellulose or polyvinylpyrrolidone and other optional ingredients known in the art to permit tableting the mixture by known methods. The tablets may, if desired, be coated using known methods and excipients which may include enteric coating using for example hydroxypropylmethylcellulose phthalate. The tablets may be formulated in a manner known to those skilled in the art so as to give a sustained release of a suitable active agent(s). Such tablets may, if desired, be provided with enteric coatings by known methods, for example by the use of cellulose acetate phthalate. Similarly, capsules, for example hard or soft gelatin capsules, containing the active agent(s) with or without added excipients, may be prepared by known methods and, if desired, provided with enteric coatings in a known manner. The contents of the capsule may be formulated using known methods so as to give sustained release of the active agent(s).

Other dosage forms for oral administration include, for example, aqueous suspensions containing an active agent(s) in an aqueous medium in the presence of a non-toxic suspending agent such as sodium carboxymethylcellulose, and oily suspensions containing the active agent(s) in a suitable vegetable oil, for example arachis oil. The active agent(s) may be formulated into granules with or without additional excipients.

The granules may be ingested directly by the patient or they may be added to a suitable liquid carrier (for example, water) before ingestion. The granules may contain disintegrants, e.g. an effervescent couple formed from an acid and a carbonate or bicarbonate salt to facilitate dispersion in the liquid medium.

Nutrient Absorption Suppressants: Oral Compositions

Active agents which act on the central nervous system (CNS) to suppress appetite and, therefore, suppress nutrient absorption may be used in the present oral compositions. One major subclass of CNS appetite suppressant drugs interacts with catecholaminergic receptors in the brainstem. These include controlled drugs such as amphetamine, phenmetrazine, and diethylproprion, and over-the-counter drugs such as phenylpropanolamine. Manizidol is another CNS active drug which, although not a catecholamine, activates the central nervous system.

Other suitable active agents are drugs which promote malabsorption of nutrients through suppression of digestive enzymes. One agent in this category is Acarbose, a bacterial inhibitor of amylase and brushborder glycosidases. Another is tetrahydrolipostatin, a fungal inhibitor of lipases. These agents work by preventing digestion of carbohydrates and/or fats, thus creating an effective reduction in the number of calories absorbed, despite continued consumption.

Satiety inducing agents induce a feeling of satiety (suppress appetite) resulting in a net reduction in caloric intake following ingestion, shifting the balance of the body to enhanced lipolysis. Oleic acid and its esters are preferred satiety inducing agents.

Thermogenic Agents: Oral or Topical Compositions

Thermogenic agents, which act by promoting either metabolic activity in general or lipolytic activity in particular, may also be included in the present oral compositions. Lipolytic agents are a preferred sub-class of thermogenic agents. The catecholamine drugs discussed above have some thermogenic activity, in addition to their suppression of appetite. Thyroid hormone is also optionally used. The thermogenic agent may also include one or more of kola nut, N-acetyl-L-carnitine, cayenne extract, salicin, niacin or a derivative thereof (inducing niacinamide) or inositol hexanicotinate. N-acetyl-L-carnitine is useful in facilitating the transport of fat into mitochondria for their metabilization to generate energy. Cayenne extract stimulates the production of energy in the form of adenosine triphosphate (ATP) which, in turn, metabolizes more fat. Salicin, which is found naturally in the bark of the white willow, also has been implicated in the stimulation of thermogenesis. Niacin, also known as vitamin B-3, and its derivatives are known to induce thermogenesis and act to lower low density lipoprotein (LDL) cholesterol levels and elevate high density lipoprotein (HDL) cholesterol levels. It does so by reducing lipoprotein synthesis in the liver.

Agents which have a heating effect when applied on the skin, e.g., rubifacients, are also considered to be thermogenic agents.

Lipolytic agents are preferred thermogenic agents. A large number of active lipolytic agents may be used in the present compositions, such as asiatic acid; methylxanthines including caffeine, theophylline and aminophylline; nicotinic acid derivatives, such as α-tocopherol nicotinate or hexyl nicotinate; silicon; carnitine; coenzyme Q; escin; ruscogenin; draining, firming, lipolytic or veinotropic plant extracts; anti-glucose-uptake active agents; α-2-blocker compounds capable of blocking the α-2 receptors at the surface of adipocytes, such as ginkgo biloba; keratolytic agents, such as 5-octanoylsalicylic acid; salicylic acid; α-hydroxy acids such as lactic acid, malic acid, glycolic acid or tartaric acid or α-hydroxy acids from fruit, such as citric acid; polyethylene glycol fatty acid esters, glycerophosphatides, phosphatidylephosphates, egg yolk lecithin, oleic acid, stearic acid, palmitate, cholesterol, mono, di, and tri-glycerides, cholesterol ester, yolk lecithin containing 5 to 20% phosphatidic acid, linoleic acid, linolenic acid, lauric acid, phosphatidyl phosphate, glycerine, soy bean oil, sesame seed oil, and tromethan. Green tea solids induce lipolysis by acting on adipocyte cells, thereby reducing fat mass of the body.

Skin Temperature Method

Skin surface temperature in the region to which thermal energy is to be applied is measured as follows. A thermocouple with relatively low thermal mass is selected, such as a YSI Precision 4000 A thermometer made by Yellow Springs Instrument Company, Inc., Yellow Springs, Ohio, USA, with 400 Series flat probe, or similar. The thermocouple probe measures 1 cm across and has a conductive side which faces the skin and a coated side which faces away from the skin. The thermocouple is affixed to an area of exposed skin central to the area to which heat will be applied, e.g., the thigh, with a thin (0.5 cm) strip of adhesive tape. The thermocouple cord is taped to the thigh in several locations away from the thermocouple to keep it from pulling the thermocouple tip. The starting temperature is recorded from the device after an equilibration period of a few minutes, in ° Celsius. The treatment device is placed in contact with the skin over the thermocouple with the thermocouple in the center of the area and temperature recording begins. The device can be prewarmed or not, and can be either actively AC or DC powered (electrical, battery, etc.) or generate heat by chemical reaction (see U.S. Pat. No. 6,123,717, incorporated herein by reference). Temperature is measured and recorded at convenient intervals. One minute intervals for the first 10-15 minutes, followed by 2-5 minute intervals for the balance of the treatment time is usually suitable. The final skin temperature is the average temperature recorded in the plateau region where an equilibrium is reached between heat input and heat loss, the latter primarily due to blood perfusion in the skin. The temperature rise is expressed as the final temperature minus the initial average temperature, in ° Celsius. After treatment has been completed, skin surface temperature can continue to be monitored after removal of the device as an indication of blood flow homeostasis.

The following examples demonstrate the following for treatment of regional fat deposits including cellulite:

1. The device itself, and in combination with other devices

2. The device as a kit with topical compositions

3. Device, topical composition and oral composition as a kit or program

4. At least 1 of the elements above with a monitoring function as part of a program (%body fat monitoring, e.g.) or a business practice—home monitoring or at a spa, exercise club, etc.

As additional disclosure, the topical compositions include active agents for treating regional fat deposits including cellulite, with the object of rebuilding the dermis (stimulate collagen, revasculaturize); anti-inflammatory action; anti-histamine action; lipolysis; hormonal therapy; and/or thermogenesis.

Without wishing to be bound by theory, it is believed that the action of the device (including the device combinations) achieves its objective by one or more of the following mechanisms; biostimulation and generally enhanced cellular activity; enhanced streaming; enhanced lymphatic drainage; promotion of tissue vascularization; cavitation; and/or increased blood flow (massage, heat, etc.)

### EXAMPLE 1

A heating pad is prepared. 225 inches of coated thermal resistance wire (17.3 ohms/foot, available, for example, as 0.006 inch diameter wire from Bob Martin Co. 2209-T North Seaman Ave., So. El Monte, Calif. 91733, or http://www.bobmartinco.com/martin4.htm) is sewn to a semi-rigid nylon mesh measuring 12 inches×13.5 inches, running the wire across the width of the mesh in segments 10 inches long, and looping back every 10 inches, with about ½ inch separation between rows. The wire does not cross itself at any point on the mesh. Excess mesh is folded over the wire and both sides of the wire and mesh are wrapped with, and sewn to, a polyester nonwoven batting having a basis weight of 84 grams per square meter. The ends of the resistance wire are connected through bimetallic thermal safety switches to an A.C. power source at 120 volts. A controller integral to the power cord includes a phase fired switch to create a user-adjustable temperature control. The entire assembly is wrapped and sealed in waterproof vinyl. A covering of soft polyester is wrapped over the vinyl, and two Velcro™ straps are sewn to the covering with the 15 inch hook portion sewn along the long direction of the pad and cover, and the 24 inch loop portion of the strap extending beyond the strap edge, and a 2 inch overlap where the hook portion is sewn to the loop portion of the strap. The pad provides about 45 watts of power (maximum), or a specific power density of about 60 mW/cm² within the active portion of the pad. The pad is strapped to the stomach of an obese male with an extended Velcro™ strap, and skin surface temperature is measured. The power controller is turned onto its maximum power setting for 20 minutes, then cycled between ¾ power and maximum for another 40 minutes, and then the power is turned off and the pad is removed from the skin. Temperature is measured for another 20 minutes after removal of the pad, the subsequent increase over basal temperature indicating enhanced blood flow continuing during this period. The following skin temperature results are obtained:

| Elapsed Time | | | | | | | | | | | | | | | | | |
|---|---|---|---|---|---|---|---|---|---|---|---|---|---|---|---|---|---|
| (Minutes) | 0 | 1.5 | 3 | 4.5 | 6 | 7.5 | 9 | 10 | 12 | 14 | 16 | 18 | 20 | 25 | 30 | 35 | 40 |
| Skin surface | 29.82 | 31.5 | 33.6 | 35.4 | 37 | 38.2 | 39.1 | 39.62 | 40.44 | 40.96 | 41.16 | 41.52 | 41.66 | 40.11 | 39.52 | 41.04 | 41.44 |
| Temperature (° C.) | | | | | | | | | | | | | | | | | |

| Elapsed Time | | | | | | | | | | | | | | | | |
|---|---|---|---|---|---|---|---|---|---|---|---|---|---|---|---|---|
| (Minutes) | 45 | 50 | 55 | 60 | 61 | 62 | 63 | 64 | 65 | 66 | 68 | 70 | 72 | 75 | 80 | 90 |
| Skin surface | 40.16 | 39.44 | 40.64 | 40.82 | 36.82 | 35.18 | 34.42 | 34.18 | 34.16 | 34.28 | 34.22 | 34.24 | 34.22 | 33.6 | 33.24 | 31.5 |
| Temperature (° C.) | | | | | | | | | | | | | | | | |

A topical composition 3-formyl chromone and glycerin in the form of a skin cream is prepared by conventional methods from the following components.

| | | Weight |
|---|---|---|
| Phase | Ingredient (CTFA Name) | % |
| A | Water U.S.P. | 55.31 |
| | Disodium EDTA | 0.13 |
| | Methyl Paraben | 0.25 |
| | Glycerin | 3.00 |
| | 3-formyl chromone | 2.00 |
| | Zinc Citrate | 1.00 |
| B | Cetyl Alcohol | 0.56 |
| | Stearyl Alcohol | 2.03 |
| | Behenyl Alcohol | 0.22 |
| | Steareth-21 (Brij 721) | 0.37 |
| | Steareth-2 (Brij 72) | 1.10 |
| | Distearyldimonium chloride | 0.95 |
| | Propyl Paraben | 0.10 |
| | Polypropylene glycol-15 steareth ether (Arlamol | 3.25 |
| | E) | |
| C | Polypropylene glycol-15 steareth ether (Arlamol | 2.17 |
| | E) | |
| | Titanium dioxide | 0.75 |
| D | Citric acid | 0.19 |
| | Water U.S.P. | 22.00 |
| | 50% NaOH | 0.94 |
| E | Benzyl Alcohol | 0.50 |
| | Silicone fluid (DC Q2-1401) | 0.75 |
| | Cyclomethicone/dimethiconol—50/50 blend | 1.00 |
| | dimethicone 10 cSt | |
| | Polyethylene Low Density Beads | 1.00 |
| F | Fragrance | 0.10 |
| G | 50% NaOH | 0.33 |

Blend the A phase components with a suitable mixer (e.g., Tekmar model RW20DZM), heating while stirring to a temperature of 70-80° C. Separately, blend the B phase components with a suitable mixer and heat with mixing to melt the components. Separately, blend the C phase components and mill to obtain an acceptably smooth mixture (e.g., using a Tekmar T50 Mill). Add the C phase mixture to the B phase mixture and mix. Then add the resulting mix to the A phase mixture with mixing, cool with a cold water bath and mill, then continue stirring. Remove the combination from the bath, with continued stirring, once the temperature reaches 40° C. Separately, blend the D phase components by stirring until dissolved, then add this to the combination of A-C materials. Separately, blend the E phase components by mixing until smooth and continuous, then add this to the combination of the A-D materials. Add and mix the fragrance, then the NaOH. Adjust the pH as necessary to 5.5. Alternatively, the 3-formyl chromone can be replaced with an equivalent quantity of another flavonoid (e.g., chalcone, flavanone, isoflavone, coumarin, flavone, another chromone, dicoumarol, chromanone, chromanol, or mixtures).

After removal of the heating pad, the skin cream is applied liberally to the stomach at a rate of 2 mg composition per square centimeter of skin, and allowed to dry. The process is repeated for about 6 months, placing the heating pad each day in a different location around the abdomen, to promote regional lipolysis, thermogenesis, and regional fat reduction.

### EXAMPLE 2

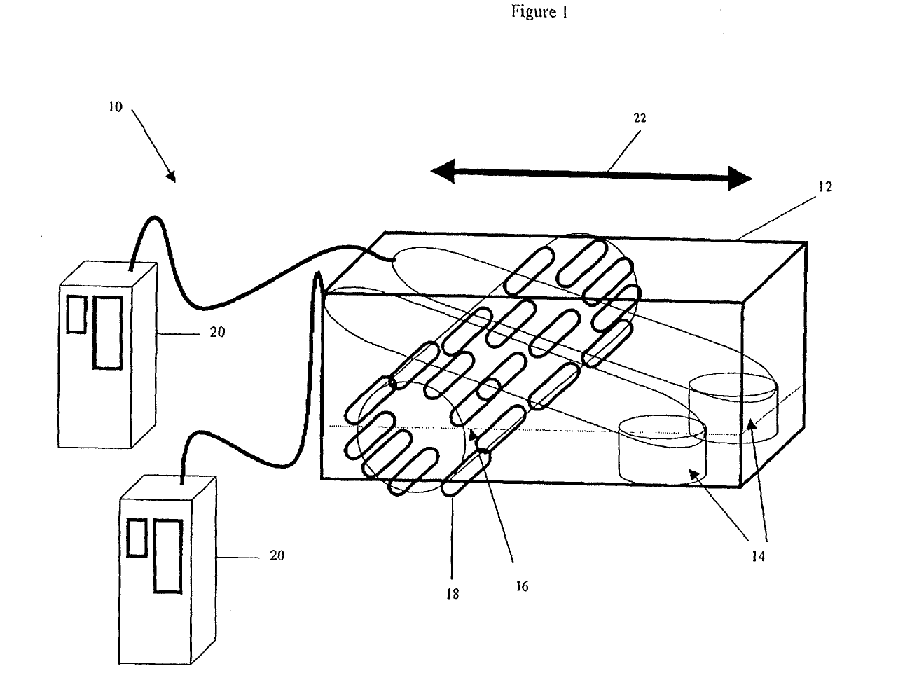

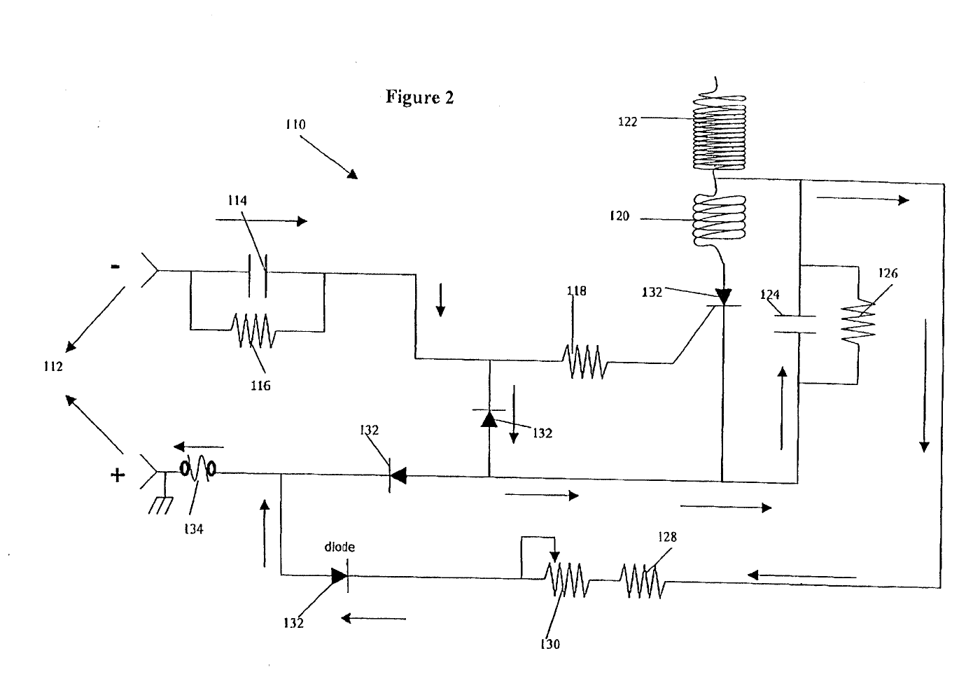

A heating pad 10 is prepared (see FIG. 1). 100 inches of coated thermal resistance wire 14 (approximately 0.8 ohms/inch, available for example as 0.008 inch diameter wire from Bob Martin Co. 2209-T North Seaman Ave., So. El Monte, Calif. 91733, or http://www.bobmartinco.com/martin4.htm) is taped to a first substrate 12, which is a segment of a dense polyester blanket measuring 5.75 inches by 9.5 inches. The wire is wrapped in non-overlapping fashion as in Example 1, with the long direction parallel to the long direction of the substrate. There are 10 lines of wire across the substrate, spaced at about 0.5 inches apart and 0.5 inches from the edge. Four additional layers of the same substrate, which is thermally insulating, are placed contiguous to the first substrate on the side away from the wires, and sewn together, to provide insulation against heat loss from one side of the pad but no insulation on the skin contact side. This arrangement is particularly beneficial in enabling low power and in facilitating therefore, the use of batteries as a power source. This arrangement, in turn, facilitates provision of a portable device. The wires are connected by conventional wire leads 16 to a lab D.C. power supply 18, for example, a Model Number 72-2075 manufactured by Tenma, operating at 26.5 volts. During operation, the pad array draws 0.33 amps, so the power can be calculated as 26.5 volts×0.33 amps=8.75 Watts. The specific power density is about 25 mW/cm², less than half the specific power density of Example 1. The array is strapped to the thigh of a woman exhibiting signs of cellulite, and skin surface temperature is measured. The YSI thermocouple is positioned between adjacent wires. The pad is placed with the wires directly contacting the skin and the insulation over the wires. After 30 minutes, the pad is moved so that a wire crosses directly over the top of the thermocouple, to determine the maximum skin surface temperature at any point under the pad, and temperature is measured for an additional 10 minutes.

The following results are obtained. Surprisingly, even with the relatively low specific power density of the pad, the skin surface temperature increases well into the therapeutic range. Also surprisingly, heat distribution is relatively even, and there are no apparent ‘hot spots’ of excessive temperature increase, nor any pain exhibited by the user. The pad is worn on different areas of the thigh and buttocks where the condition of cellulite is evident, once or twice per day for 30 to 60 minutes in each location, for a period of 6 months. The appearance of cellulite is evaluated monthly to monitor progress.

| Elapsed Time | | | | | | | | | | | | | | | |
|---|---|---|---|---|---|---|---|---|---|---|---|---|---|---|---|
| (Minutes) | 0 | 1 | 2 | 3 | 4 | 5 | 6 | 7 | 8 | 9 | 10 | 12 | 14 | 16 | 18 |
| Skin surface | 32.12 | 32.88 | 33.86 | 34.68 | 35.4 | 36 | 36.56 | 37.02 | 37.44 | 37.82 | 38.14 | 38.72 | 39.14 | 39.46 | 39.7 |
| Temperature (° C.) | | | | | | | | | | | | | | | |

| Elapsed Time | | | | | | | | | | | | | | |
|---|---|---|---|---|---|---|---|---|---|---|---|---|---|---|
| (Minutes) | 20 | 25 | 30 | 30.1 | 31 | 32 | 33 | 34 | 35 | 36 | 37 | 38 | 39 | 40 |
| Skin surface | 39.84 | 39.86 | 39.82 | 39.84 | 40.16 | 40.26 | 40.3 | 40.3 | 40.32 | 40.32 | 40.34 | 40.38 | 40.38 | 40.36 |
| Temperature (° C.) | | | | | | | | | | | | | | |

A skin cream emulsion comprising a blend of niacinamide, glycerin and isoflavone is prepared by conventional methods from the following components and then applied after removal of the heating pad.

| Ingredient | Weight % |
|---|---|
| Silicone fluid (Dow Corning DC 345) | 15.0 |
| Silicone fluid (Dow Corning DC 3225C) | 2.5 |
| Silicone fluid (Goldschmidt Abil We09) | 2.5 |
| Water | 66.4 |
| Niacinamide | 5.0 |
| Unsubstituted flavanone | 5.0 |
| Tetrasodium EDTA | 0.1 |
| Benzyl alcohol | 0.3 |
| Methyl paraben | 0.2 |
| Glycerin | 3.0 |

Form the water phase in a suitable vessel charged with the water as follows: add the glycerin and then niacinamide to the water with stirring. Add to this mixture with stirring the methyl paraben dissolved in the benzyl alcohol. Add to this mixture with stirring the EDTA. Form the silicone phase in a separate suitable vessel by adding and stirring together the silicone fluids and the unsubstituted flavanone. Add the water phase to the silicone phase slowly with stirring to form the emulsion. Apply the resulting composition to a subject's skin which exhibits signs of cellulite at the rate of 2 mg composition/cm² skin once or twice daily for a period of at least 3-6 months to improve skin surface texture. Alternatively, the unsubstituted flavanone can be replaced with an equivalent quantity of another polycyclic compound (e.g., chalcone, another flavanone, isoflavone, coumarin, flavone, chromone, dicoumarol, chromanone, chromanol, triterpenoid (e.g., betulinic acid), sterol (e.g., stigmasterol), or mixtures thereof).

### EXAMPLE 3

A battery powered heating pad is prepared. The heating pad of Example 2 is prepared with the exception that 100 inches of 0.057 ohm/cm wire is substituted (available from SIS Filaments & Heater Wires (www.sisweb.com/ms/sis/wire3.htm)). The wire is connected to two, six-cell rechargeable NiMH batteries in series, which deliver 14.8 volts theoretical, dropping to about 12 volts during discharge. The batteries are rated at 6500 milliamp-hours each to discharge (Panasonic D-cell NiMH available at www.digi-key.com). The batteries are connected to a belt clip, allowing the user to move freely about during therapeutic heat treatment. The array provides nearly 10 Watts of power and nearly 8 hours of continuous use before recharging of the batteries is necessary.

A skin cream containing niacinamide, theophylline, glycerin, caffeine and panthenol is prepared by combining and mixing the ingredients of each column using conventional technology.

| Ingredient | % Weight |
|---|---|
| Glycerine | 6.933 |
| Niacinamide | 14.00 |
| Theophylline | 1.500 |
| Caffeine | 0.500 |
| Permethyl 101A¹ | 3.000 |
| Sepigel² | 2.500 |
| Q2-1403³ | 2.000 |
| Isopropyl Isostearate | 1.330 |
| Arlatone 2121⁴ | 1.000 |
| Cetyl Alcohol CO-1695 | 0.720 |
| SEFA Cottonate⁵ | 0.670 |
| Tocopherol Acetate | 0.500 |
| Panthenol | 0.500 |
| Adol 62⁶ | 0.480 |
| Kobo Titanium Dioxide | 0.400 |
| Sodium Hydroxide 50% Aqueous | 0.0150 |
| Fiery 5⁷ | 0.150 |
| Disodium EDTA | 0.100 |
| Glydant Plus⁸ | 0.100 |
| Myrj 59⁹ | 0.100 |
| Emersol 132¹⁰ | 0.100 |
| Color | 0.00165 |
| Purified Water | q.s. to 100 |

¹Isohexadecane, Presperse Inc., South Plainfield, NJ;
²Polyacrylamide(and)C13-14 Isoparaffin(and)Laureth-7, Seppic Corporation, Fairfield, NJ;
³dimethicone(and)dimethiconol, Dow Corning Corp., Midland, MI;
⁴Sorbitan Monostearate and Sucrococoate, ICI Americas Inc., Wilmington, DE;
⁵Sucrose ester of fatty acid, Procter and Gamble, Cincinnati, OH;
⁶Stearyl alcohol, Procter and Gamble, Cincinnati, OH;
⁷Fiery, Procter and Gamble, Cincinnati, OH;
⁸DMDM Hydantoin (and) Iodopropynyl Butylcarbamate, Lonza Inc., Fairlawn, NJ;
⁹PEG-100 Stearate, ICI Americas Inc., Wilmington, DE;
¹⁰Stearic acid, Henkel Corp., Kankakee, IL.

The composition is applied to the thighs of a woman showing signs of cellulite at a rate of 2 mg of composition per square centimeter of skin, at least once per day. The heating pad is worn at least one hour per day. Treatment is continued for 3 months or until there is a visible reduction in the appearance of cellulite and treatment is no longer needed.

### EXAMPLE 4

A kit is prepared comprising an oral composition, a topical composition, and a disposable heat pad.

A disposable heating pad is prepared by the method described in U. S. Pat. No. 6,123,717. The pad comprises a heating portion measuring about 7.5 inches×5 inches, which contains 16 elliptically shaped disks with 1.5 inch×0.875 inch axes, comprising the air-activated thermal component. The length of the entire pad is 32 inches, and it has an extended flexible component to wrap around the body, with a hook and loop type closure system to hold it in place. A commercial embodiment of the pad is sold by the Procter & Gamble Company, Cincinnati, Ohio, USA as a S/M size ThermaCare Therapeutic Heat Wrap. The wrap is opened and immediately wrapped around the thigh of a woman exhibiting signs of cellulite, and skin surface temperature is measured. After 60 minutes, the disposable pad is removed, and skin surface temperature measurement is continued an additional 40 minutes, the temperature elevation in the absence of the heating pad demonstrating additional therapeutic benefit. The following results are obtained.

| Elapsed Time | | | | | | | | | | | | | | | | | | |
|---|---|---|---|---|---|---|---|---|---|---|---|---|---|---|---|---|---|---|
| (Minutes) | 0 | 1.5 | 3 | 4.5 | 6 | 8 | 10 | 12 | 15 | 18 | 21 | 24 | 27 | 30 | 35 | 40 | 45 | 50 |
| Skin Surface | 31.8 | 31.96 | 32.28 | 32.44 | 32.62 | 32.84 | 33.12 | 33.44 | 33.68 | 35 | 35.44 | 35.8 | 36.04 | 36.2 | 36.44 | 36.72 | 36.86 | 36.76 |
| Temperature (° C.) | | | | | | | | | | | | | | | | | | |

| Elapsed Time | | | | | | | | | | | | | | | | | |
|---|---|---|---|---|---|---|---|---|---|---|---|---|---|---|---|---|---|
| (Minutes) | 55 | 60 | 61 | 62 | 63 | 64 | 65 | 66 | 68 | 72 | 76 | 80 | 84 | 88 | 92 | 96 | 100 |
| Skin Surface | 36.94 | 37.24 | 34.94 | 34.16 | 33.68 | 33.4 | 33.3 | 33.18 | 32.74 | 32.16 | 31.8 | 31.54 | 31.32 | 31.06 | 30.88 | 30.76 | 30.56 |
| Temperature (° C.) | | | | | | | | | | | | | | | | | |

An oral composition is prepared as follows. A single packet of a dry instant beverage mix is blended with 8.0 grams of oleic acid. The dry instant beverage mix is sold by Nestle® Carnation® as French Vanilla flavored Instant Breakfast™ Nutritional Energy Drink (or similar) and comprises about 36.3 grams of dry instant mix. The dry mix contains these ingredients: nonfat dry milk, maltodextrin, sugar, cellulose gum, natural and artificial vanilla flavor, dicalcium phosphate, magnesium hydrochloride, sodium ascorbate, ferric orthophosphate, vitamin E acetate, niacinamide, copper gluconate, zinc oxide, calcium pantothenate, manganese sulfate, vitamin A palmitate, pyridoxine hydrochloride, thiamin mononitrate, folic acid, biotin, phylloquinone, vitamin B12. The oleic acid is stirred into the dry mixture. A dietary supplement beverage is prepared from the resulting mixture by stirring into 8 fluid ounces of skim milk. The dietary supplement beverage is consumed as breakfast (in place of other food for breakfast) as part of an extended program to reduce the appearance of cellulite. The beverage (oral composition) induces a feeling of satiety resulting in a net reduction of caloric intake over the course of the day the beverage is consumed, shifting the metabolic balance of the body to enhanced lipolysis. The local action of the heat pad promotes lipolysis from the adipocytes of the thigh region in order to preferentially reduce the size of those cells and tissues, mitigating the appearance of cellulite. In addition to daily application of the heat pad for 1 hour, and daily consumption of the oleate containing beverage, the topical composition of Example 3 is applied daily to the entire thigh and buttocks region. Over a 6 month period, total body fat is monitored by bioelectric impedance using an Omron HBF 300 Body Fat Analyzer (BodyTrends.com, 1-800-549-1667, http://bodytrends.com/omhbf300.htm) demonstrating a systemic reduction in body fat which accompanies an improvement in the appearance of cellulite due to the local treatments.

### EXAMPLE 5

A kit is prepared comprising an oral composition in the form of a dietary supplement, a topical composition, and a heat pad.

A topical composition is prepared which is an emulsion containing retinyl palmitate, a dermal strengthening compound. The emulsion is prepared by first creating the water phase and then creating the oil phase. After both phases are created, they are mixed together and retinyl palmitate is added. The water phase is made by first weighing deionized water into a beaker and, with mixing at high speed, slowly adding carboxy polymer. EDTA and ascorbic acid are then added to the mixture and mixing is continued until well-dissolved, about 40 minutes. The water phase is then heated to 80° C., at which time propylene glycol is added. To make the oil phase, all ingredients of the oil phase are weighed and added together in a separate beaker, heating to 80° C. with mixing until homogeneous. The oil phase is then slowly poured into the water phase with mixing. Sodium hydroxide is added at 80° C. in order to adjust the pH of the emulsion. After mixing for ten minutes, the emulsion is cooled to 45° C. Retinyl palmitate is then added to the emulsion and the emulsion is mixed until homogeneous. The procedure is carried out under yellow light and under a nitrogen blanket so as to minimize exposure to oxygen.

| | Content |
|---|---|
| Ingredient | (% W/W) |
| Carboxyvinyl polymer | 0.300 |
| Propylene glycol | 5.00 |
| Methylparaben | 0.15 |
| Ascorbic Acid | 0.10 |
| Glyceryl monostearate | 5.00 |
| Cetanol | 1.00 |
| Stearyl alcohol | 0.50 |
| White Petrolatum | 1.50 |
| BHT | 0.05 |
| Propylparaben | 0.10 |
| Butylparaben | 0.05 |
| Cetyl palmitate | 1.00 |
| C12-C15 Alkyl Benzoate | 4.00 |
| Benzyl alcohol | 0.30 |
| Ethyl alcohol | 4.00 |
| Disodium EDTA | 0.05 |
| Retinyl palmitate | 0.30 |
| Sodium Hydroxide (10%) | to adjust pH to 8.0 |
| Water | QS |

Capsules are prepared. 5.0 g pharmaceutical grade caffeine are dry blended with 10.0 g dried kava root (standardized to provide a kavalactone content of about 30% by wt and containing the full spectrum of lactones found in the kava plant) and 20.0 g dried green tea solids (prepared by freeze drying the boiled water extract of green tea leaves and containing the full range of green tea polyphenols, i.e., catechins and epicatechins). The dried mixture is filled into gelatin capsules, 900 mg per gel cap. The gelatin capsules are administered orally to a woman exhibiting signs of cellulite and obesity throughout the day for a period of 12 weeks, three tablets per day taken 45 minutes before each meal. The tablets increase metabolism, hence lipolytic metabolic activity. Twice daily, about 5 grams of the topical composition is applied to her thigh and buttocks region. Once per day, the heat pad of Example 1 is worn on at least one thigh for at least 1 hour per location. Optionally, the woman can also wear Bioskin Compression Shorts (available from The Brace Store, Brownsville, Tex., USA, http://www.thebracestore.com/thigh_supports.html) to promote drainage of the lymph and temporarily reduce thigh girth.

### EXAMPLE 6

An infrared heating device for emitting infrared at a wavelength of 2000 to 15000nm is applied to the thigh. The device comprises a 7.5 Watt bulb housed in a parabolic metallized reflector with a flat plastic cover approximately 15 mm away from the surface of the bulb at its closest point. The cover measures about 85 mm diameter. A commercial embodiment is made by the Lumiscope Company, Inc., Edison, N.J., 08837, USA, and is called an Infralume Curved Handle Heat Lamp. The device is placed 14 mm above the skin of the thigh with YSI thermocouple in place to measure skin surface temperature. The following results are obtained. Heating of a single spot occurs rapidly, so the device is moved around each thigh for a period of 20 minutes.

| | Skin Surface |
|---|---|
| Elapsed Time | Temperature |
| (minutes) | (° C.) |
| 0 | 31.36 |
| 1 | 39.6 |
| 2 | 44.18 |
| 3 | 47.2 |
| 4 | 49.1 |

After heating, the topical composition of Example 5 is applied to the skin. The dual treatments are applied daily for a period of 3 months to reduce the appearance of cellulite.

### EXAMPLE 7

A disposable wrap 10 is prepared which provides heat and magnetic energy simultaneously, as is illustrated in FIG. 2. A disposable thermal wrap 14 is prepared which comprises individual heat cells of oxygen-activated exothermic disks contained between two continuous nonwoven layers. One-inch diameter and 0.5 inch thick heat disks are prepared and compacted in the manner disclosed in U.S. Pat. No. 6,020,040. Twenty-four disks are prepared and sealed in pockets between a layer of impermeable film and a layer of film having an oxygen permeability of 3 cc O2 /min./5 cm2 (at 21° C., 1 ATM), spaced 3 inches apart center-to-center in a hexagonal packing array. Pockets are created between the heat cells by heat sealing an 80 gsm polyester nonwoven segment measuring about 1.5 inch square on 3 edges only to the sleeve between the heat cells. 1-inch diameter neodymium magnets having a field strength of 1,000 Gauss are inserted. A nonwoven, flexible sleeve 12 is prepared by heat sealing polypropylene nonwoven (80 gsm) to the thermal wrap 14, with an elastic edge and hook-and-loop type closure system 16 to enable the thermal wrap 14 to be encircled around the thigh. The sleeve 12 may be sized such as to provide a tight fit to the area of application, thereby applying therapeutic compression to the regional fat deposits.

### EXAMPLE 8

A disposable wrap is prepared which provides heat and electrostatic energy simultaneously. A disposable thermal wrap is prepared which comprises individual heat cells of oxygen-activated exothermic disks contained between two continuous nonwoven layers. One-inch diameter and 0.5 inch thick heat disks are prepared and compacted in the manner disclosed in U.S. Pat. No. 6,020,040. Twenty-four disks are prepared and sealed in pockets between a layer of impermeable film and a layer of film having an oxygen permeability of 3 cc O2 /min./5 cm2 (at 21° C., 1 ATM), spaced 3 inches apart center-to-center in a hexagonal packing array.

An electret is prepared which is a polypropylene substrate having a basis weight of about 88 grams per square meter and having a surface charge of at least about 7,000 volts. The process for preparing this electret is detailed for example in U.S. Pat. No. 4,142,521. One such commercially available electret is the Pain T. E. M. Therapeutic Electro Membrane available by ordering at the web site http://www.paintem.com. Surface charge is measured by an electrostatic voltmeter, for example the Trek Model 523 hand-held electrostatic voltmeter, measured with the electrode in contact with the substrate. The electrostatic voltmeter is available from Trek, Inc., at hftp://www.trekinc.com/523sp.htrn. The electret is placed on the inside of the heat-generating wrap (the skin contacting side) and the combination wrap is wrapped around the thigh for 6 hours to provide a combination of electrostatic potential energy and heat to the skin simultaneously. A new combination wrap is used daily, and treatments are applied daily for 2 months to visibly reduce the signs of regional fat deposits including cellulite.

### EXAMPLE 9

A neoprene flexible sleeve is prepared. A light patch array is fabricated using Gallium Arsenide Phosphide on Gallium Phosphide red light emitting diodes (LEDs) which illuminate maximally at about 635 nm. Spectral analysis is verified with a spectrometer, for example Ocean Optics SD2000 High Sensitivity Fiber Optic Spectrometer with OOiBase32 PC software, from Ocean Optics, Inc. Agilent Technologies HLMP-1340 T-1 diodes are used. Each of the diodes measures approximately 3 mm diameter with transparent lenses and a 45 degree viewing angle. An individual diode delivers about 0.10 milliwatts (mW) optical power at 1.85 volts; 0.16 mW optical power at 1.95 volts; and 0.32 mW optical power at 2.14 volts, drawing 8.0, 15.1 and 30.3 milliamps (mA) current, respectively, at the specified voltages. Optical power is measured with a multifunctional optical power meter, for example an Oriel OPM Model 70310 with enhanced UV Silicone Detector, a 1 cm square array, and the LED positioned as close as possible to the detector (8 mm). The LEDs are connected in parallel by soldering to a standard rigid printed circuit board with 0.1 inch (0.254 cm) grid using a diode density of 25 two-pin diodes per square inch (6.45 cm²) (i.e., 50% of PC board capacity). The PC board measures 6 inches (15.24 cm) by 4 inches (10.16 cm), with 438 diodes covering an inner 5 inch (12.7 cm) by 3.5 inch (8.89 cm) rectangular area of the board. Two six-cell rechargeable NiMH batteries are connected in series to deliver power through a DC-DC switching converter to reduce voltage to approximately 2.0 volts, and the voltage is trimmed using an adjustment circuit and potentiometer to deliver 1.95 volts to the array, measured across each diode. The array has an optical power of about 0.60 mW/cm². A small, battery powered fan is affixed to the back of the array to remove excess heat generated during use. The array is affixed to an elastic neoprene sleeve (5 mm thick) measuring about 25 inches (63.5 cm) long by 8.5 inches (21.59 cm) wide that has two, 2-inch (5.08 cm) wide elastic straps that extend another 10 inches (25.4 cm) in length. To the exposed skin contact area of the sleeve, 112 inches of a coated, fine resistance heating wire is sewn. The wire is 0.014 ohm/cm, 0.040 inch diameter 80% Ni/20% Cr wire available for example from SIS Filaments & Heater Wires (www.sisweb.com/ms/sis/wire3.htm). A heat resistant, 3 mm thick heat resistant nonwoven is laid over the resistance wire, and a soft, polyester nonwoven over the heat resistant nonwoven, the latter two with a cutout so as not to interfere with the light transmission part of the sleeve. The nonwovens are sewn to attach to the sleeve at all edges and around the center cutout. Power is supplied to the resistance wire by the same NiMH battery packs running in parallel to the light diode array, connected in parallel through a switching controller with a 50% on duty cycle. The batteries deliver 14.4 volts theoretical which drops to about 12 volts during discharge, are connected in series and are rated at 6500 milliamp-hours each to discharge. The straps are attachable and detachable to the bulk of the sleeve by a hook and loop type fastening system, to affix the sleeve to the thigh while concurrently allowing therapeutic compression to be applied. A rectangular hole is cut in the center of the neoprene sleeve, and straps located at its edge to allow the light patch array to sit within the sleeve. Wires connect the array to the power supply. The power supply and battery are contained in a pouch with a hook to attach to the belt or waistband of the user, so the sleeve can be worn while the user is active. The sleeve is attached to the thigh of a user to treat regional fat deposits including cellulite, the power supply switched on, and the user resumes normal activity for a period of between 0.5 and 2 hours, applying about 1 to 4 Joules/cm²(J/cm²). After this period, the sleeve is rotated or moved to apply energy to a different site, moved to the other leg, or removed. By applying energy to different sites, the entire thigh is treated with light and therapeutic compression. After about 3 hours of continuous use, the batteries are recharged to prepare them for another cycle. The skin cream of, for example, Example 3, is applied to the thighs of a woman showing signs of cellulite at a rate of 2 mg of composition per square centimeter of skin, at least once per day. Treatment with the skin cream and light/compression is continued for 3 months or until there is a visible reduction in the appearance of cellulite and treatment is not needed any more.

The above described arrangements are merely illustrative of the principles of the present invention. Other modifications or adaptations may occur to those skilled in the art, without departing from the spirit and scope of the present invention.

What is claimed is:

1. A method for the treatment of a selected area of the skin and/or subcutaneous tissue, and in particular for the cosmetic treatment of skin conditions such as regional fat deposits including cellulite, the method comprising heating the selected area to a sustained skin temperature of about 32 to about 50° C. for a desired period of time, using a device comprising either a heat source capable of conductively heating the selected area or, alternatively, an infra-red source for emitting infra-red with a wavelength from about 700 nm to about 15000 nm; and administering, simultaneously or sequentially in either order, a composition containing an active agent selected from the group comprising a skin active; a nutrient absorption suppressant; or a thermogenic agent or a lipolytic agent, or a mixture thereof, the composition being either a topical composition for application to the selected area, or an area adjacent thereto, or an oral composition.

2. A method according to claim 1 wherein the thermogenic agent or the lipolytic agent is selected from the group consisting of caffeine, theophylline and aminophylline.

3. A method according to claim 1 wherein the nutrient absorption suppressant is selected from the group consisting of oleic acid and its esters.

4. A method according to claim 1 wherein the skin active is selected from the group consisting of niacinamide, retinoid and mixtures thereof.

5. A method according to claim 1 wherein the sustained skin temperature is about 32 to about 45° C.

6. A method according to claim 1 wherein the desired period of time is about 20 seconds to about 24 hours.

7. A method according to claim 1 comprising the additional step of exposing the selected area, or an area adjacent thereto, to a source of at least one alternative energy form selected from the group comprising light, electrotherapy, active massage, static magnets, compression, and combinations of two or more thereof.

8. A method according to claim 7 wherein the selected area is simultaneously exposed to the heat and to the alternative energy form source.

9. A method according to claim 7 wherein the selected area is sequentially exposed, in either order, to the heat and to the alternative energy form source.

10. A method according to claim 7 wherein the alternative energy form is electromagnetic radiation having a wavelength of from approximately 400 nm to approximately 1500 nm.

11. A method according to claim 10 wherein the alternative energy form source is electromagnetic radiation in the form of one or more LEDs.

12. A method according to claim 7 wherein the alternative energy form is electrotherapy and the method additionally comprises electrostatically applying, from a storage medium, a current between the storage medium and the selected area, or an area adjacent thereto.

13. A method according to claim 7 wherein the alternative energy form source is static magnets and the method further comprises exposing the selected area, or an area adjacent thereto, to a static magnetic field having a field intensity in the range 100 Gauss to 2000 Gauss.

14. A kit for the treatment of skin and/or subcutaneous tissue, and in particular for the treatment or prevention of regional fat deposits including cellulite in a selected area, the kit including:
i. a device for maintaining a sustained skin temperature of about 32 to about 50° C. for a desired period of time, the device comprising either a heat source capable of conductively heating the selected area or, alternatively, an infra-red source for emitting infra-red with a wavelength from about 700 nm to about 15000 nm;
ii. a composition containing an active agent selected from the group comprising a skin active; a nutrient absorption suppressant; or a thermogenic agent or a lipolytic agent, or a mixture thereof, the composition being either a topical composition for application to the selected area or an area adjacent thereto, or an oral composition; and
iii. instructions for their simultaneous or sequential use in either order.

15. A kit according to claim 14 wherein the thermogenic agent or the lipolytic agent is selected from the group consisting of green tea solids, caffeine, theophylline, aminophylline and mixtures thereof.

16. A kit according to claim 14 wherein the nutrient absorption suppressant is selected from the group consisting of oleic acid, oleic acid esters and mixtures thereof.

17. A kit according to claim 14 wherein the skin active is a retinoid.

18. A kit according to claim 14 wherein the sustained skin temperature is about 32 to about 45° C., preferably about 34 to about 45° C.

19. A kit according to claim 14 wherein the desired period of time is about 20 seconds to about 24 hours.

20. A kit according to claim 14 comprising the additional step of exposing the selected area, or an area adjacent thereto, to a source of at least one alternative energy form selected from the group comprising light, electrotherapy, active massage, static magnets, compression, and combinations of two or more thereof.

21. A kit according to claim 20 wherein the selected area is simultaneously exposed to the heat and to the alternative energy form source.

22. A kit according to claim 20 wherein the selected area is sequentially exposed, in either order, to the heat and to the alternative energy form source.

23. A kit according to claim 20 wherein the alternative energy form is electromagnetic radiation having a wavelength of from approximately 400 nm to approximately 1500 nm.

24. A kit according to claim 23 wherein the alternative energy form source is electromagnetic radiation in the form of one or more LEDs.

25. A kit according to claim 20 wherein the alternative energy form is electrotherapy and the method additionally comprises electrostatically applying, from a storage medium, a current between the storage medium and the selected area, or an area adjacent thereto.

26. A kit according to claim 20 wherein the alternative energy form source is static magnets and the method further comprises exposing the selected area, or an area adjacent thereto, to a static magnetic field having a field intensity in the range 100 Gauss to 2000 Gauss.

27. A device for the treatment of skin and/or subcutaneous tissue, and in particular for the treatment or prevention of regional fat deposits including cellulite in a selected area, the device being adapted for maintaining a sustained skin temperature of about 32 to about 50° C. for a desired period of time, the device comprising either a heat source capable of conductively heating the selected area or, alternatively, an infra-red source for emitting infra-red with a wavelength from about 700 nm to about 15000 nm; the device also being adapted for domestic use or unsupervised clinic use.

28. A device according to claim 27 wherein the device additionally comprises a portable power source, preferably a battery.

29. A device according to claim 27 wherein the device has a skin contacting surface and, on a surface opposed from the skin contacting surface, is provided with insulation material.

30. A device according to claim 27 wherein the device is shaped and dimensioned for portability.
